(19) United States
(12) Patent Application Publication
(10) Pub. No.: US 2024/0120026 A1
(43) Pub. Date: Apr. 11, 2024

(54) METHOD AND DEVICE FOR EXTRACTING SOMATIC MUTATIONS FROM SINGLE-CELL TRANSCRIPTOME SEQUENCING DATA

(71) Applicant: Zhejiang University, Hangzhou (CN)

(72) Inventors: Ning SHEN, Hangzhou (CN); Tianyun ZHANG, Hangzhou (CN)

(21) Appl. No.: 18/460,039

(22) Filed: Sep. 1, 2023

(30) Foreign Application Priority Data
Sep. 30, 2022 (CN) 202211212629.3

(51) Int. Cl. G16B 20/20; G16B 40/20

(52) U.S. Cl. CPC G16B 20/20; G16B 40/20

(57) ABSTRACT

A method for extracting somatic mutations from single-cell transcriptome sequencing data includes processing single-cell transcriptome raw sequencing data with a first comparison and identification method to obtain a plurality of first somatic mutation sites, processing the single-cell transcriptome raw sequencing data with a second comparison and identification method to obtain a plurality of second somatic mutation sites, integrating the plurality of first somatic mutation sites and the plurality of second somatic mutation sites to obtain a plurality of candidate somatic mutation sites, and performing mutation screening on the plurality of candidate somatic mutation sites to obtain final somatic mutation sites.

### CROSS-REFERENCE TO RELATED APPLICATION

This application claims priority to Chinese Application No. 202211212629.3, filed Sep. 30, 2022, the entire content of which is incorporated herein by reference.

### FIELD OF TECHNOLOGY

Embodiments of the present disclosure relate to a technical field of transcriptome data analysis, and in particular, to a method and device for extracting somatic mutations from single-cell transcriptome sequencing data.

### BACKGROUND

Cancer is a series of changes caused by mutations in the genome of cells, acting on the genome, epigenome, transcriptome and other levels of cells. The tissue heterogeneity and rapid evolution of cancer cells are the key points and research difficulties in tumor development and treatment tolerance. In recent years, single-cell transcriptome technology has been rapidly developed and widely used, and a series of progress has been made in the heterogeneity of transcriptional expression profiles and the evolution of drug resistance in tumor tissues. However, there are still many difficulties in the detection and analysis of genomic mutations such as somatic mutations at the single-cell genome level due to the development of single-cell genome sequencing technology. It is even more difficult to simultaneously detect the genome and transcriptome at the single-cell level, so as to achieve genotype-to-phenotype research at the single-cell level.

Single-cell RNA sequencing (scRNA-seq) data experiments cover relatively few genomic regions at the single-cell level, resulting in the sparsity of detectable mutations. A large number of spurious signals and noise signals will be introduced during the experiments, which further increases the difficulty in detecting somatic mutations with high precision in this data type.

### SUMMARY

Based on the above-mentioned situation in the existing technologies, the purpose of the embodiments of the present disclosure is to provide a method and device for extracting somatic mutations from single-cell transcriptome sequencing data. The direct and high-precision extraction of somatic mutation information from single-cell RNA sequencing (scRNA-seq) data is realized by providing a high-precision bioinformatics algorithm framework.

In order to achieve the above purpose, according to one aspect of the present disclosure, a method for extracting somatic mutations from single-cell transcriptome sequencing data is provided, including:
processing single-cell transcriptome raw sequencing data with a first comparison and identification method to obtain a plurality of first somatic mutation sites;
processing the single-cell transcriptome raw sequencing data with a second comparison and identification method to obtain a plurality of second somatic mutation sites;
integrating the plurality of first somatic mutation sites and the plurality of second somatic mutation sites to obtain a plurality of candidate somatic mutation sites; and
performing mutation screening on the plurality of candidate somatic mutation sites to obtain final somatic mutation sites.

Further, processing single-cell transcriptome raw sequencing data with a first comparison and identification method to obtain a plurality of first somatic mutation sites includes: comparing the single-cell transcriptome raw sequencing data with reference genome data using a first comparison mode to obtain a first comparison information record file;
labeling the first comparison information record file; and performing correction and annotation the first comparison information record file that has been labeled to obtain the plurality of first somatic mutation sites; where the correction includes sequence correction and base quality correction, and the annotation includes annotation of functional effects on encoded proteins and annotation of a database for germline mutations and RNA editing.

Further, processing the single-cell transcriptome raw sequencing data with a second comparison and identification method to obtain a plurality of second somatic mutation sites includes:
comparing the single-cell transcriptome raw sequencing data with reference genome data using a second comparison mode; and
obtaining the plurality of second somatic mutation sites with a second identification mode from results obtained by the comparing.

Further, integrating the plurality of first somatic mutation sites and the plurality of second somatic mutation sites to obtain a plurality of candidate somatic mutation sites includes: filtering the plurality of first somatic mutation sites to obtain a plurality of filtered first somatic sites; and
comparing the plurality of filtered first somatic mutation sites with the plurality of second somatic mutation sites to obtain the plurality of candidate somatic mutation sites.

Further, filtering the plurality of first somatic mutation sites includes: excluding mutation sites located in a preset exclusion region among the first somatic mutation sites; and
performing site screening with the database after annotating remaining first somatic mutation sites to obtain the filtered first somatic mutation sites.

Further, comparing the plurality of filtered first somatic mutation sites with the plurality of second somatic mutation sites to obtain the plurality of candidate somatic mutation sites includes:
regarding sites shared by the plurality of filtered first somatic mutation sites and the plurality of second somatic mutation sites as the candidate somatic mutation sites for each single cell; and
regarding sites only existing in the plurality of filtered first somatic mutation sites or the plurality of second somatic mutation sites as noise sites for each single cell.

Further, performing mutation screening on the plurality of candidate somatic mutation sites to obtain final somatic mutation sites includes:
screening the obtained candidate somatic mutation sites and noise sites by using a first quality condition, a second quality condition and a first reproduction condition.

Further, candidate somatic mutation sites that meet all of the first quality condition, the second quality condition and the first reproduction condition are regarded as the final somatic mutation sites;
regarding noise sites that do not meet any of the first quality condition, the second quality condition and the first reproduction condition as final noise sites; and
regarding remaining candidate somatic mutation sites and noise sites as undetermined candidate sites.

Further, the method also includes:
using the final somatic mutation sites and final noise sites as training data to train a mutation extraction model; and
making predictions on the undetermined candidate sites with a trained mutation extraction model to screen somatic mutation sites among the undetermined candidate sites.

Further, the mutation extraction model includes a first logistic regression model and a second logistic regression model;
the first logistic regression model is established by using a detection quality value of the mutation sites, coverage of reads, a probability of each genotype after normalization, the number of bases supported by each of two bases, and a ratio of mutant alleles to the number of all reads at the sites; and
the second logistic regression model is established by using a mutation type of the mutation sites, information of one base before and after the mutation sites, and information of a mutation spectrum.

Further, output results of the first logistic regression model and the second logistic regression model are integrated to obtain a prediction result of the mutation extraction model with the following formula:

P(pos_{classifier})=½Σ_{ΣPϵ(P}_{_{seq}}_{,P}_{_{qual}}₎wP

wherein w is an integration coefficient, when P≥0.5, w=1, and otherwise, w=0; P( ) represents a probability function that the candidate sites are real mutations, pos_{classifier} represents the candidate sites, P_{qual} represents the output result of the first logistic regression model for a same candidate mutation site, and P_{seq} represents the output result of the second logistic regression model for the same candidate mutation site.

According to a second aspect of the present disclosure, a device for extracting somatic mutations from single-cell transcriptome sequencing data is provided, including:
a first comparison and identification module used to process single-cell transcriptome raw sequencing data with a first comparison and identification method to obtain a plurality of first somatic mutation sites;
a second comparison and identification module used to process the single-cell transcriptome raw sequencing data with a second comparison and identification method to obtain a plurality of second somatic mutation sites;
a candidate somatic cell mutation site acquisition module used to integrate the plurality of first somatic mutation sites and the plurality of second somatic mutation sites to obtain a plurality of candidate somatic mutation sites; and
a mutation screening module used to perform mutation screening on the plurality of candidate somatic mutation sites to obtain final somatic mutation sites.

According to a third aspect of the present disclosure, an electronic device is provided, including a memory, a processor, and executable instructions stored in the memory and executable on the processor, the processor implementing the method according to the first aspect of the disclosure when executing a program.

According to a fourth aspect of the present disclosure, a computer-readable storage medium having computer-executable instructions stored thereon is provided, the executable instructions implementing the method according to the first aspect of the disclosure when executed by the processor.

To sum up, the embodiments of the present disclosure provide a method and device for extracting somatic mutations from single-cell transcriptome sequencing data, the method including: processing single-cell transcriptome raw sequencing data with a first comparison and identification method to obtain a plurality of first somatic mutation sites; processing the single-cell transcriptome raw sequencing data with a second comparison and identification method to obtain a plurality of second somatic mutation sites; integrating the plurality of first somatic mutation sites and the plurality of second somatic mutation sites to obtain a plurality of candidate somatic mutation sites; and performing mutation screening on the plurality of candidate somatic mutation sites to obtain final somatic mutation sites. The embodiments of the present disclosure have the following beneficial technical effects relative to the existing technologies:
(1) The technical solutions of the embodiments of the present disclosure, while retaining the maximum number of real mutations as much as possible, minimize the deviation caused by the comparison and mutation extraction algorithms themselves, and effectively remove the misidentified noise caused by the extraction algorithm itself, by comparing the single-cell transcriptome raw sequencing data and reference genome data, and reusing the two comparison methods.
(2) The technical solutions of the embodiments of the present disclosure filters the compared data to eliminate interference, and further performs mutation screening, and sets the quality conditions and reproduction condition respectively to realize the screening, thereby effectively reducing the influence of noise at each stage on the results, which solves the problem of more noise in single-cell data.
(3) The technical solutions of the embodiments of the present disclosure can also construct a mutation extraction model by combining a logistic regression model, and make further predictions on the obtained undetermined candidate sites, which not only ensures the precision of the entire extraction method, but also improves the sensitivity of the extraction method.

### BRIEF DESCRIPTION OF THE DRAWINGS

FIG. 1 is a flowchart of a method for extracting somatic mutations from single-cell transcriptome sequencing data provided by an embodiment of the present disclosure;

FIG. 2 is a flowchart of a method for extracting somatic mutations from single-cell transcriptome sequencing data provided by a second embodiment of the present disclosure;

FIG. 3 is a block structural diagram of a device for extracting somatic mutations from single-cell transcriptome sequencing data provided by an embodiment of the present disclosure;

FIG. 4 is a schematic structural diagram of an electronic device provided by an embodiment of the present disclosure.

### DESCRIPTION OF THE EMBODIMENTS

In order to make the purpose, technical solutions and advantages of the present disclosure clearer, the present disclosure will be further described in detail below with reference to the detailed description and the accompanying drawings. It should be understood that these descriptions are only exemplary and are not intended to limit the scope of the disclosure.

It should be noted that, unless otherwise defined, technical terms or scientific terms used in one or more embodiments of the present disclosure shall have common meanings understood by those with ordinary skill in the art to which this disclosure belongs. The terms “first,” “second” and similar terms used in one or more embodiments of the present disclosure do not denote any order, quantity, or importance, but are merely used to distinguish the various components. “Contain,” “include,” or “comprise” and similar words mean that the elements or things appearing before the word encompass the elements or things recited after the word and their equivalents, but do not exclude other elements or things. “Connect” or “link” and similar terms are not limited to physical or mechanical connections, but may include electrical connections, whether direct or indirect. “Up,” “Down,” “Left,” “Right,” etc. are only used to represent a relative positional relationship, and when the absolute position of the described object changes, the relative positional relationship may also change accordingly.

At present, it is difficult to simultaneously detect the genome and transcriptome at the single-cell level, so as to achieve genotype-to-phenotype research at the single-cell level. First, from the experimental aspect, the experimental method of combining targeted sequencing/genotyping on the basis of single-cell transcriptome sequencing, or the method of combining single-cell transcriptome sequencing with traditional bulk whole exome sequencing (bulk WES) or bulk whole genome sequencing (bulk WGS) to analyze base mutations at the single-cell transcriptome level has been reported successively. In addition, computational and analytical methods for tumor evolution and lineage tracing studies based on integrating single-cell RNA sequencing (scRNA-seq) data with traditional bulk whole exome or genome sequencing have also been reported. However, such methods require a large number of biological samples, a perfect experimental design and experimental technology, but have very limited detection sensitivity, so such multi-omics data based on the same sample are rare. The corresponding algorithm is also difficult to be widely used.

In contrast, bioinformatics algorithms that directly extract genomic mutation information carried by mRNA from single-cell RNA sequencing (scRNA-seq) data are more efficient. Since many somatic mutations at the genomic DNA level will be carried by the corresponding transcript mRNA, somatic mutations that are transcribed to the mRNA level and carried by high expression are more likely to play a role in cancer cells than somatic mutations that are silenced and not expressed. Moreover, directly detecting highly expressed somatic mutations in single-cell RNA sequencing (scRNA-seq) data can simultaneously extract genomic mutation information and gene expression information in the same single cell without additional experiments, truly realizing genotype-to-phenotype research at the single-cell level.

The embodiments of the present disclosure provide a method and device for extracting somatic mutations from single-cell transcriptome sequencing data, which solves the technical problems that the single-cell RNA sequencing (scRNA-seq) data experiments themselves cover fewer genomic regions at the single-cell level, resulting in the sparsity of detectable mutations, and a large number of spurious signals and noise signals will be introduced during the experiments.

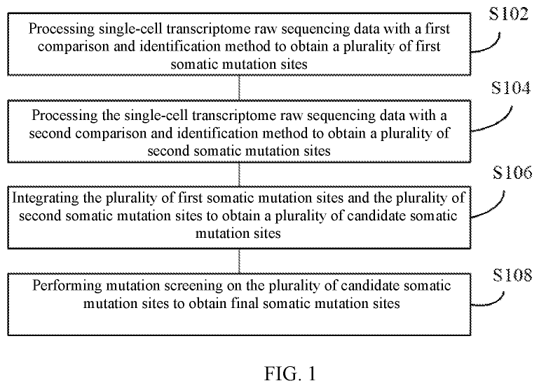

The technical solutions of the present disclosure will be described in detail below with reference to the accompanying drawings. In a first embodiment of the present disclosure, a method for extracting somatic mutations from single-cell transcriptome sequencing data is provided. FIG. 1 shows a flowchart of the method, including the following steps:
S102, processing single-cell transcriptome raw sequencing data with a first comparison and identification method to obtain a plurality of first somatic mutation sites.
S104, processing the single-cell transcriptome raw sequencing data with a second comparison and identification method to obtain a plurality of second somatic mutation sites.

In the above steps S102 and S104, comparing the single-cell transcriptome raw sequencing data and reference genome data with a first comparison and identification method and a second comparison and identification method respectively can identify mutation sites awaiting subsequent identification. The reference genome data can be downloaded from existing databases. The goal of the above steps is to retain the maximum number of real mutations as much as possible in the candidate gene pool. In order to minimize the deviation caused by the comparison and mutation identification algorithms themselves, two different comparison and variation detection algorithms are adopted, i.e. a first comparison and identification method and a second comparison and identification method, and through subsequently comparing the results of the two groups of different comparison and identification methods, the deviations of the algorithms themselves are minimized, so that the misidentified noise caused by the identification algorithm itself can be effectively removed. The first comparison and identification method includes a comparison and identification method based on quality features, and database screening containing information such as germline mutations and RNA editing; the second comparison method includes a comparison and identification method based on long noisy read aligner and a mixture distribution model. Compared with the traditional aligner, in the embodiment of the present disclosure, software that can compare data with long reads (for example, 100 MB) is selected to realize the comparison based on long noisy read aligner, and the software can also effectively deal with noise information contained in long reads.

In the step S102, processing single-cell transcriptome raw sequencing data with a first comparison and identification method to obtain a plurality of first somatic mutation sites can include the following steps:
S1021, comparing the single-cell transcriptome raw sequencing data with reference genome data using a first comparison mode to obtain a first comparison information record file. The first comparison mode, for example, can use the STAR TWO-PASS mode to compare the raw sequencing data (STAR is dedicated comparison software for single-cell RNA sequencing (scRNA-seq) data) to obtain a preliminary comparison information record file (BAM file).
S1022, labeling the first comparison information record file. On the basis of the first comparison information record file, the read group information can be added and repetitions can be labeled through Picard, where Picard is a software package of the types such as SAM/BAM/VCF for processing high-throughput test data. The sequencing data used in the embodiments of the present disclosure are paired-end sequencing data. In the paired-end sequencing data, there are two files, read1 and read, which respectively represent the sequencing data of the two ends in the paired-end sequencing. This step is used to label which paired-end sequencing file the information in the first comparison information record file (BAM file) comes from.
S1023, performing correction and annotation the first comparison information record file that has been labeled to obtain the plurality of first somatic mutation sites. In this embodiment, for example, the SplitNCigarReads tool specially developed for RNA sequencing (RNA-seq) data is used to separate sequences that fall on exons, remove N wrong bases (where N represents non-determined bases), and remove sequences in intronic regions. The base quality of sequences is adjusted by the BaseRecalibrator and ApplyBQSR base quality correction tools provided by GATK. GATK HaplotypeCaller can provide preliminary variation detection. In this process, only sites with Phred-scale quality scores greater than 20 will be considered as high-quality variations and retained, thus correcting the above information record file. During the annotation process, for the sites in the above information record file, SnpEff (SnpEff is protein function annotation software) is first used to annotate and predict whether the mutations will affect protein-encoding genes; then RNAediting and dbSNP databases or user-given germline mutation information is used respectively for further annotation; finally mutations located within 6 bases from the end of the reads are removed. In sequencing data, the sequenced base reads all have a fixed length. The bases located at the edge of the sequencing read length are very likely to be mutated due to noise pollution from sequencing instruments or the experimental process. To improve the accuracy of the identification, the bases at the end of the read length are removed at the end of this step.

Through the above-mentioned first comparison and identification method, the preliminary detection of variation sites is realized, which can provide more information for subsequent analysis.

In step S104, processing the single-cell transcriptome raw sequencing data with a second comparison and identification method to obtain a plurality of second somatic mutation sites can include the following process: comparing the single-cell transcriptome raw sequencing data with reference genome data using a second comparison mode, and obtaining the plurality of second somatic mutation sites with a second identification mode from results obtained by the comparing. In this step, in order to minimize the deviation of the detection algorithm itself, another completely different comparison mode is used. For example, in this embodiment, the minimap2 comparison algorithm can be used as the second comparison mode. Compared with the STAR comparison algorithm, the minimap2 comparison algorithm can process RNA sequencing (RNA-seq) data of long noisy read aligner. After the comparison is completed, Strelka, an algorithm for mutation detection, can be used as the second identification mode to determine the candidate pool of mutation sites. Strelka is used to perform somatic mutation detection on the bam file output by minimap2. The principle is mainly to establish a mixture distribution model by comparing the output bam file with the reference genome data again. By using the mixture distribution model to estimate the probability of belonging to variant mutations and the probability of belonging to noise for each input site to be predicted, the mutation rate and the noise rate are estimated, thereby realizing somatic mutation detection.
S106, integrating the plurality of first somatic mutation sites and the plurality of second somatic mutation sites to obtain a plurality of candidate somatic mutation sites. In this step, the first somatic mutation sites and the second somatic mutation sites obtained based on the above comparison step are integrated to achieve further screening and identification, which can specifically include the following steps:
S1061, filtering the plurality of first somatic mutation sites to obtain a plurality of filtered first somatic sites. The somatic mutations identified by the first comparison method (i.e. the first somatic mutation sites) are filtered and screened, and only the mutations that are located in the exon region and are not germline mutations in the candidate mutation pool are retained to ensure that the influence of germline mutations is excluded; the mutation sites obtained by screening are then compared with the mutations identified in the second comparison method (i.e. the second somatic mutation sites), and the shared part is retained to minimize the error of the algorithm itself. It can be achieved by the following steps: excluding mutation sites located in a preset exclusion region among the first somatic mutation sites; and performing site screening with the database after annotating remaining first somatic mutation sites to obtain the filtered first somatic mutation sites. For example, for the first somatic mutation sites, the mutation sites located in the chrM and GL regions are excluded; then ANNOVAR (ANNOVAR is software for annotating SNP and other variation sites) is used to annotate each first somatic mutation site based on ensGene data, and filter again to ensure that each first somatic mutation site is located in the exon region. Ensembl is a bioinformatics research program that aims to develop software capable of automatic annotation and maintenance of eukaryotic genomes. The database provided by this project is ensGene. Based on the common mutations in the population in the gnomAD30 database, the candidate mutations are filtered to exclude the interference of the common mutations, and then the RNAedit data of the human RNA editing database based on the human reference genome hg38 is used to remove the RNA editing sites from the first somatic mutation sites. The interference of germline mutations on the limited identification of somatic mutations can be excluded by the filtering steps described above.
S1062, comparing the plurality of filtered first somatic mutation sites with the plurality of second somatic mutation sites, and retaining the part shared by the two as candidate somatic mutation sites for real somatic mutations: regarding sites shared by the plurality of filtered first somatic mutation sites and the plurality of second somatic mutation sites as the candidate somatic mutation sites for each single cell; and regarding sites only existing in the plurality of filtered first somatic mutation sites or the plurality of second somatic mutation sites as noise sites for each single cell.
S108, performing mutation screening on the plurality of candidate somatic mutation sites to obtain final somatic mutation sites. In this step, mutations in a single cell are further screened based on data quality, and high-quality mutations are retained to eliminate the influence of some noise. Moreover, for a single mutation, if the mutation appears repeatedly in multiple cells of the same sample, the mutation is considered to be a highly credible somatic mutation, which can be very effective in eliminating noise. Because the noise is more of a random occurrence, it cannot be reproduced across multiple cells. Mutations satisfying the above conditions are defined as final somatic mutations with high confidence. Mutations that only appear in a single identification algorithm and fail to meet the quality and reproducibility conditions are defined as noise. We defined remaining somatic mutations that only meet some of the conditions as an undetermined group for subsequent modeling testing. The mutation screening process can screen the obtained candidate somatic mutation sites and noise sites by using the first quality condition, the second quality condition and the first reproduction condition:
regarding the candidate somatic mutation sites that meet all of the first quality condition, the second quality condition and the first reproduction condition as the final somatic mutation sites; regarding the noise sites that do not meet any of the first quality condition, the second quality condition and the first reproduction condition as the final noise sites; and regarding the remaining candidate somatic mutation sites and noise sites as undetermined candidate sites.

In this step, mutation screening based on real somatic mutation sites needs to meet the following conditions in terms of quality information:
the first quality condition: the detection quality of the variation information is not less than 30. The sequencing depth of this site is greater than the set parameter (default is 3), and the result of evaluating that the current variation may be strand bias by Fisher's test (Fisher's test is an exact test used to test whether the results of a random experiment support the hypothesis for a certain random experiment) is not greater than 30.

The second quality condition: if there are BaseQRankSum (BaseQRankSum means to compare the quality of bases that support variation and bases that support the reference genome, and a negative value means that the quality of bases that support variation is lower than that of the bases that support the reference genome) and ClippingRankSum, the value is required to be not less than −2.33 and not greater than 2.33, and the values of MQRankSum and ReadPosRankSum are not less than −2.33 and not greater than 2.33. Hard clipping means that if the read length cannot be compared with the reference genome, the read length will be deleted. ClippingRankSum means to perform a base rank sum test on the data where the deleted base is located at the reference base and the mutated base; MQRankSum means to perform a rank sum test on comparison quality located at the reference base and the mutated base; and ReadPosRankSum means to perform a rank sum test on the relative positions of the reference base and the mutated base in the read length. The first reproduction condition: the number of occurrences of each mutation site in all cells of a single sample is counted. If the number of occurrences of the mutation site is not less than 3 or 5% of the total number of cells and not more than 80% of the total number of cells, the mutation site is considered to be a real somatic mutation site and meets the first reproduction condition.

In the above steps, the interference of non-exonic regions and germline mutations is first excluded through annotations and related databases, and then the quality screening conditions and mutation reproduction times conditions are used to effectively reduce the impact of noise at each stage on the results, thereby solving the problem of more noise in single-cell data.

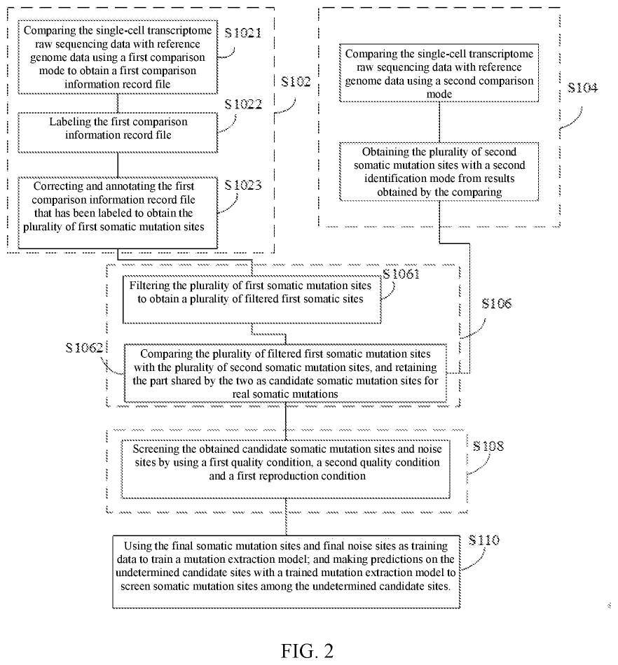

In the second embodiment provided by the present disclosure, the method can further include the following steps:
S110, using the final somatic mutation sites and final noise sites as training data to train a mutation extraction model; and making predictions on the undetermined candidate sites with a trained mutation extraction model to screen somatic mutation sites among the undetermined candidate sites. Using the trained model for prediction, as an optional embodiment, aims to improve the sensitivity of the extraction method. FIG. 2 shows a flowchart of an extraction method provided by a second embodiment. Given that credible somatic mutations and noise have been identified in the steps above, a supervised learning model can be employed to build the model. In addition, for a single mutation, quality-related features and sequence-related features are included after the annotation in the above steps. Due to the different attributes of the two types of features, different from the previous hybrid modeling, in this embodiment of the present disclosure, the models are trained separately for the two types of features, so as to prevent any one type of feature from having a great influence on the overall model. When building a model based on sequence-related features, the concept of a mutation spectrum is introduced, i.e. different types of mutations occurring at different rates in different types of cell lines or cancer types. Therefore, by building a model of this type of features, specific modeling of different types of cancers or cell line samples is achieved. Model training based on quality-related features is more focused on modeling the commonality of different types of cancers or cell line samples. Finally, by integrating the above two models, a joint logistic regression model is formed to make predictions in the groups that cannot be determined. The sparsity of data is another difficulty in extracting somatic mutations directly from single-cell data. After filtering based on the conditions provided in the above steps in the embodiment of the present disclosure, the number of mutation sites obtained is relatively small. In order to further improve the sensitivity of the algorithm, for cancer tissues and cell line samples that contain many somatic mutations, the joint logistic regression model can be used. With the supervised learning model, the model is trained based on known somatic mutations and noise information before making predictions on data that cannot be classified. Since there is a certain degree of imbalance in the number of real somatic mutations and noise, the problem of overall data imbalance can be adjusted by oversampling before training the model.

In this embodiment of the present disclosure, the mutation extraction model mainly includes two independent logistic regression models. The first logistic regression model is based on quality features and the second logistic regression model is based on sequence features. The sequencing data of the mutation sites can be used to detect the quality value, the coverage of reads (the coverage of the sequencing data for each site), the probability of each genotype after normalization, and the number of bases supported by each of the two bases (the number of reference bases and mutated bases), and the proportion of mutated alleles to the number of all reads at the site to build the first logistic regression model. A genotype refers to the general name of all gene combinations of a biological individual, and the probability of each genotype after normalization refers to the proportion of various genotypes after normalizing the data; and the variant allele score refers to the ratio of the coverage depth of reads supporting the reference/alternative allele at a certain site in the genome to the total reads coverage depth of the site. The second logistic regression model can be established by using the mutation type of the mutation sites, the information of one base before and after the mutation sites, and the information of the mutation spectrum. The quality characteristics involved in the above-mentioned model establishment process can be directly obtained in the file of the mutation sites obtained in the preceding steps. Sequence features can be annotated and obtained by the R package MutationalPatterns (R package MutationalPatterns is software developed based on the R language to annotate the mutation map of the mutation sites according to the mutation position and other information) based on the mutation position information obtained in the previous steps.

Due to the small number of training samples, in order to avoid overfitting, a regularization penalty term can be introduced. For the first logistic regression model, since it is necessary to avoid the influence of outliers on the model for quality features, L1 regularization can be selected. For the second logistic regression model, in order to avoid the model being overly focused on common mutation types while ignoring relatively rare mutation types, L2 regularization is selected. In the embodiment of the present disclosure, output results of the first logistic regression model and the second logistic regression model are integrated to obtain a prediction result of the mutation extraction model with the following formula:

P(pos_{classifier})=½Σ_{ΣPϵ(P}_{_{seq}}_{,P}_{_{qual}}₎wP

where w is an integration coefficient, when P≥0.5, w=1, and otherwise, w=0. P( ) represents a probability function that the candidate sites are real mutations, pos_{classifier} represents the candidate sites, P_{qual} represents the output result of the first logistic regression model for a same candidate mutation site; and P_{seq} represents the output result of the second logistic regression model for the same candidate mutation site. The output results of the first logistic regression model and the second logistic regression model are, for the same candidate mutation sites, the probability that the sites are predicted to be real mutations.

Through the above steps, while ensuring the precision of the entire extraction method, the sensitivity of the extraction method is also improved.

In order to evaluate the precision of the overall method, the methods of the embodiments of the present disclosure is applied to 8 cell lines and simulated tissue test datasets, respectively.

TABLE 1

| | Precision | | | | | | |
|---|---|---|---|---|---|---|---|
| | | Second | | | | | |
| | | embodiment | | | | | |
| | | (including | | | | | |
| | | the mutation | | | | | |
| | First | extraction | | | | | |
| Cell line | embodiment | model) | Enge_2017 | Maynard_2020 | Varscan | Hovestadt_2019 | BCFTools |
| JURKAT SMART-seq+ | 0.9 | 0.9 | 0.03 | 0.056 | 0.041 | 0.047 | 0.014 |
| JURKAT Target-seq | 0.862 | 0.865 | 0.026 | 0.061 | 0.039 | 0.047 | 0.011 |
| SET2 SMART-seq+ | 0.723 | 0.785 | 0.0013 | 0.004 | 0.0018 | 0.0025 | 0.00063 |
| SET2 Target-seq | 0.62 | 0.7 | 0.00065 | 0.004 | 0.0012 | 0.0021 | 0.00036 |
| LNCaP_0 hr | 0.63 | 0.764 | 0.0065 | 0.079 | 0.01 | 0.013 | 0.0036 |
| LNCaP_12 hr | 0.65 | 0.78 | 0.0065 | 0.082 | 0.009 | 0.011 | 0.0023 |
| K562 primer | 0.7 | 0.667 | 0.00075 | 0.088 | 0.00084 | 0.002 | 0.000453 |
| K562 no primer | 0.625 | 0.625 | 0.000886 | 0.1138 | 0.00072 | 0.0016 | 0.000446 |

Table 1 shows the data of somatic mutation extraction precision when applying the methods of the first embodiment and the second embodiment of the present disclosure to 8 cell lines and simulated tissue datasets, respectively. Among them, Enge_2017, Maynard_2020, Varscan, Hovestadt_2019, and BCFTools are the somatic mutation extraction methods used in the existing technologies.

For the simulated tissue test datasets, the test datasets contain infant and toddler data. Somatic mutations of multiple different cancer cell lines are added to infant and toddler tissue samples through computational simulation for data simulation. Then the extraction methods provided by the embodiments of the present disclosure and the other five existing extraction methods are applied to the test datasets. Table 2 shows the comparison of the precision results of the data simulation. The results show that, compared with other methods in the existing technologies, the extraction method provided by the first embodiment of the present disclosure can achieve stable and high-precision somatic mutation detection. Moreover, in the simulated data containing more somatic cell mutations, the method provided by the second embodiment of the present disclosure can achieve higher sensitivity than the extraction method provided by the first embodiment of the present disclosure, but in the simulated data containing fewer somatic mutations, the precision of the method provided by the second embodiment of the present disclosure is lower than that of the method provided by the first embodiment of the present disclosure. Therefore, for data containing fewer somatic mutations, only applying the extraction method provided by the first embodiment of the present disclosure can identify somatic mutations with high precision, and provide accurate directions for subsequent cancer or drug target research. For samples containing more somatic mutations, applying the extraction method provided by the second embodiment of the present disclosure can improve the sensitivity of the algorithm to a certain extent on the premise of ensuring high precision.

TABLE 2

| Cell | Number | First | Second | | | | | |
|---|---|---|---|---|---|---|---|---|
| line | of cells | embodiment | embodiment | Enge_2017 | Maynard_2020 | Varscan | Hovestadt_2019 | BCFTools |
| HCT15 | 50 | 0.9773 | 0.9731 | 0.012 | 0.7267 | 0.020 | 0.0186 | 0.0090 |
| HCT15 | 100 | 0.9573 | 0.9526 | 0.0084 | 0.6543 | 0.157 | 0.0203 | 0.0067 |
| HCT15 | 150 | 0.9358 | 0.9343 | 0.0066 | 0.6083 | 0.130 | 0.0201 | 0.0054 |
| HCT15 | 200 | 0.9121 | 0.912 | 0.0055 | 0.5711 | 0.0112 | 0.0189 | 0.0045 |
| HCT15 | 250 | 0.8871 | 0.8854 | 0.0048 | 0.5390 | 0.0102 | 0.0179 | 0.0039 |
| HCT15 | 300 | 0.8556 | 0.8565 | 0.0044 | 0.5184 | 0.0095 | 0.0174 | 0.0035 |
| HCT15 | 331 | 0.8364 | 0.8391 | 0.0042 | 0.5044 | 0.0093 | 0.0169 | 0.0033 |
| MCC13 | 50 | 0.956 | 0.9561 | 0.0053 | 0.2545 | 0.0091 | 0.0073 | 0.0039 |
| MCC13 | 100 | 0.9079 | 0.9085 | 0.0037 | 0.1994 | 0.007 | 0.0082 | 0.0030 |
| MCC13 | 150 | 0.8667 | 0.8705 | 0.0030 | 0.1656 | 0.0059 | 0.0082 | 0.0024 |
| MCC13 | 200 | 0.8295 | 0.8313 | 0.0025 | 0.1463 | 0.0051 | 0.0080 | 0.0020 |
| MCC13 | 250 | 0.789 | 0.7968 | 0.0022 | 0.1301 | 0.0047 | 0.0078 | 0.0018 |
| MCC13 | 300 | 0.7366 | 0.7452 | 0.0020 | 0.1207 | 0.0044 | 0.0076 | 0.0016 |
| MCC13 | 331 | 0.7088 | 0.7201 | 0.0019 | 0.1105 | 0.0042 | 0.0073 | 0.0015 |
| KCL22 | 50 | 0.9344 | 0.9143 | 0.0042 | 0.0082 | 0.0072 | 0.0072 | 0.0031 |
| KCL22 | 100 | 0.8758 | 0.8675 | 0.0029 | 0.0091 | 0.0053 | 0.0074 | 0.0023 |
| KCL22 | 150 | 0.8211 | 0.8077 | 0.0022 | 0.0078 | 0.0043 | 0.0069 | 0.0018 |
| KCL22 | 200 | 0.7671 | 0.7636 | 0.0018 | 0.0071 | 0.0037 | 0.0064 | 0.0015 |
| KCL22 | 250 | 0.7216 | 0.7215 | 0.0016 | 0.0063 | 0.0033 | 0.0061 | 0.0013 |
| KCL22 | 300 | 0.6539 | 0.6643 | 0.0015 | 0.0058 | 0.0031 | 0.0059 | 0.0012 |
| KCL22 | 331 | 0.6242 | 0.6309 | 0.0014 | 0.0056 | 0.0031 | 0.0057 | 0.0011 |
| PA137 | 50 | 0.9737 | 0.8525 | 0.0017 | 0 | 0.0026 | 0.0025 | 0.0012 |
| PA137 | 100 | 0.9538 | 0.81 | 0.0012 | 0 | 0.0020 | 0.0026 | 0.0009 |
| PA137 | 150 | 0.962 | 0.8362 | 0.0010 | 0 | 0.0017 | 0.0026 | 0.0008 |
| PA137 | 200 | 0.9468 | 0.8209 | 0.0008 | 0 | 0.0015 | 0.0026 | 0.0006 |
| PA137 | 250 | 0.9514 | 0.8214 | 0.0007 | 0 | 0.0013 | 0.0024 | 0.0006 |
| PA137 | 300 | 0.9386 | 0.753 | 0.0006 | 0 | 0.0012 | 0.0024 | 0.0005 |
| PA137 | 331 | 0.9381 | 0.7665 | 0.0006 | 0 | 0.0012 | 0.0023 | 0.0005 |
| HEC59 | 50 | 0.9231 | 0.7619 | 0.0012 | 0 | 0.0015 | 0.0016 | 0.0009 |
| HEC59 | 100 | 0.9615 | 0.7857 | 0.0009 | 0 | 0.0013 | 0.0016 | 0.0007 |
| HEC59 | 150 | 0.8947 | 0.5542 | 0.0007 | 0 | 0.0012 | 0.0016 | 0.0006 |
| HEC59 | 200 | 0.8776 | 0.5607 | 0.0007 | 0 | 0.0011 | 0.0015 | 0.0005 |
| HEC59 | 250 | 0.8909 | 0.4571 | 0.0006 | 0 | 0.0010 | 0.0014 | 0.0005 |
| HEC59 | 300 | 0.8966 | 0.4748 | 0.0006 | 0 | 0.0010 | 0.0014 | 0.0004 |
| HEC59 | 331 | 0.9016 | 0.4091 | 0.0005 | 0 | 0.0010 | 0.0014 | 0.0004 |
| JURKAT | 50 | 0.9524 | 0.7778 | 0.0009 | 0.0041 | 0.0015 | 0.0012 | 0.0007 |
| JURKAT | 100 | 0.963 | 0.6511 | 0.0007 | 0.0031 | 0.0013 | 0.0013 | 0.0006 |
| JURKAT | 150 | 0.8889 | 0.4861 | 0.0006 | 0.0026 | 0.0011 | 0.0013 | 0.0005 |
| JURKAT | 200 | 0.875 | 0.4742 | 0.0005 | 0.0024 | 0.0010 | 0.0013 | 0.0004 |
| JURKAT | 250 | 0.8824 | 0.4167 | 0.0005 | 0.0021 | 0.0009 | 0.0013 | 0.0004 |
| JURKAT | 300 | 0.8868 | 0.4508 | 0.0004 | 0.0020 | 0.0009 | 0.0012 | 0.0003 |
| JURKAT | 331 | 0.5455 | 0.3774 | 0.0004 | 0.0019 | 0.0009 | 0.0012 | 0.0003 |

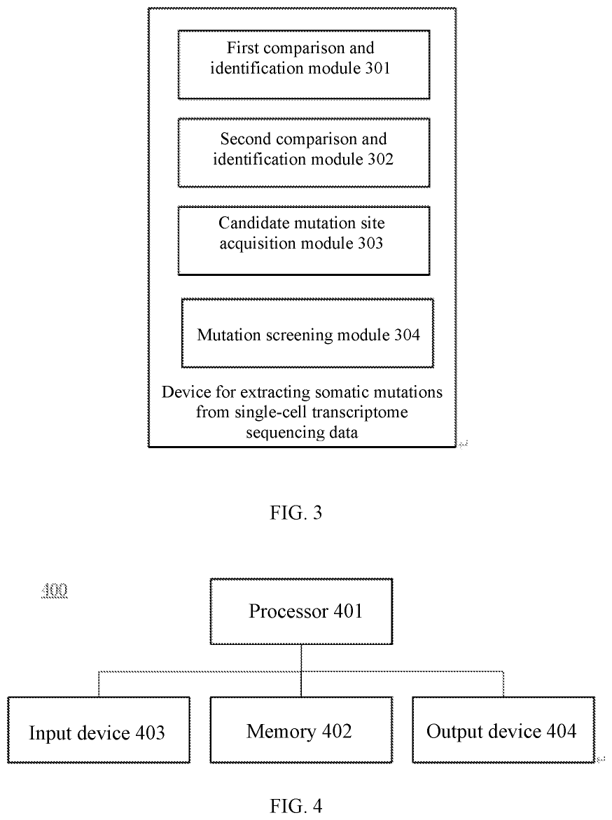

The third embodiment of the present disclosure also provides a device for extracting somatic mutations from single-cell transcriptome sequencing data. FIG. 3 shows a block structural diagram of the device, and the device includes:
a first comparison and identification module 301 used to process single-cell transcriptome raw sequencing data with a first comparison and identification method to obtain a plurality of first somatic mutation sites;
a second comparison and identification module 302 used to process the single-cell transcriptome raw sequencing data with a second comparison and identification method to obtain a plurality of second somatic mutation sites;
a candidate somatic cell mutation site acquisition module 303 used to integrate the plurality of first somatic mutation sites and the plurality of second somatic mutation sites to obtain a plurality of candidate somatic mutation sites; and
a mutation screening module 304 used to perform mutation screening on the plurality of candidate somatic mutation sites to obtain final somatic mutation sites.

The specific process for each module in this embodiment of the present disclosure to realize its function is the same as each step of the method for extracting somatic mutations from single-cell transcriptome sequencing data in the above-mentioned embodiment of the present disclosure, and the repeated description thereof will be omitted here.

In a fourth embodiment of the present disclosure, an electronic device is also provided, including a memory, a processor, and executable instructions stored in the memory and executable on the processor, the processor implementing the method according to the above-mentioned embodiment of the present disclosure when executing a program. FIG. 4 is a schematic structural diagram of an electronic device 400 provided by this embodiment of the present disclosure. As shown in FIG. 4, the electronic device 400 includes: one or more processors 401 and a memory 402; and computer-executable instructions stored in the memory 402, where the executable instructions cause the processors 401 to execute the method for extracting somatic mutations from single-cell transcriptome sequencing data in the above-mentioned embodiment when executed by the processors 401. The processors 401 can be a central processing unit (CPU) or other form of processing unit with data processing capabilities and/or instruction execution capabilities, and can control other components in the electronic device to perform desired functions. The memory 402 can include one or more computer program products, where the computer program products can include various forms of computer-readable storage media, such as a volatile memory and/or non-volatile memory. The volatile memory can include, for example, random access memory (RAM) and/or cache, and the like. The non-volatile memory may include, for example, read-only memory (ROM), hard disk, flash memory, and the like. One or more computer program instructions can be stored on the computer-readable storage media, and the processors 401 can execute the program instructions to implement the steps in the method for extracting somatic mutations from single-cell transcriptome sequencing data according to the above embodiment of the present disclosure and/or other desired functions. In some embodiments, the electronic device 400 can further include: an input device 403 and an output device 404 interconnected by a bus system and/or other forms of connecting mechanisms (not shown in FIG. 4). For example, when the electronic device is a stand-alone device, the input device 403 can be a communication network connector for receiving the collected input signal from an external movable device. In addition, the input device 403 can further include, for example, a keyboard, a mouse, a microphone, and the like. The output device 404 can output various information to the outside, and for example, it can include, for instance, a display, a speaker, a printer, and a communication network and a remote output device connected thereto.

In an embodiment of the present disclosure, a computer-readable storage medium is also provided, having a computer program stored thereon, where the computer program implements the steps in the method according to the above-mentioned embodiment of the present disclosure when executed by the processor. A computer-readable storage medium can employ any combination of one or more readable media. The readable media can be readable signal media or readable storage media. The readable storage media, for example, can include but not limited to electrical, magnetic, optical, electromagnetic, infrared, or semiconductor systems, apparatuses or devices, or a combination of any of the above. More specific examples (a non-exhaustive list) of the readable storage media include: electrical connections having one or more wires, portable disks, hard disks, random access memories (RAM), read-only memories (ROM), erasable programmable read-only memories (EPROM or flash memory), optical fibers, portable compact disk read-only memories (CD-ROM), optical storage devices, magnetic storage devices, or any suitable combination of the above.

It should be understood that the processors in the embodiment of the present disclosure can be central processing units (CPU), and the processors can also be other general-purpose processors, digital signal processors (DSP), application specific integrated circuits (ASIC), field programmable gate arrays (FPGA) or other programmable logic devices, discrete gate or transistor logic devices, discrete hardware components, and the like. General-purpose processors can be microprocessors or the processors can be any conventional processor, and the like.

To sum up, the embodiments of the present disclosure relate to a method and device for extracting somatic mutations from single-cell transcriptome sequencing data, the method including: processing single-cell transcriptome raw sequencing data with a first comparison and identification method to obtain a plurality of first somatic mutation sites; processing the single-cell transcriptome raw sequencing data with a second comparison and identification method to obtain a plurality of second somatic mutation sites; integrating the plurality of first somatic mutation sites and the plurality of second somatic mutation sites to obtain a plurality of candidate somatic mutation sites; and performing mutation screening on the plurality of candidate somatic mutation sites to obtain final somatic mutation sites. The technical solutions of the embodiments of the present disclosure, while retaining the maximum number of real mutations as much as possible, minimize the deviation caused by the comparison and mutation extraction algorithms themselves, and effectively remove the misidentified noise caused by the extraction algorithm itself, by comparing the single-cell transcriptome raw sequencing data and reference genome data, and reusing the two comparison methods. The compared data are filtered to eliminate interference, and further performs mutation screening, and sets the quality conditions and reproduction condition respectively to realize the screening, thereby effectively reducing the influence of noise at each stage on the results, which solves the problem of more noise in single-cell data. The technical solutions of the embodiments of the present disclosure can also construct a mutation extraction model by combining a logistic regression model, and make further predictions on the obtained undetermined candidate sites, which not only ensures the precision of the entire extraction method, but also improves the sensitivity of the extraction method.

It should be understood that the discussion of any of the above embodiments is only exemplary, and is not intended to imply that the scope of the present disclosure (including the claims) is limited to these examples; under the spirit of the present disclosure, the technical features in the above embodiments or different embodiments can also be combined, the steps can be implemented in any order, and there are many other variations of the various aspects of the one or more embodiments of the disclosure described above, which for the sake of brevity have not been presented in detail. The above-mentioned detailed description of the present disclosure are merely used to illustrate or explain the principle of the present disclosure, but not to limit the present disclosure. Therefore, any modifications, equivalent replacements, improvements, and the like made without departing from the spirit and scope of the present disclosure should be included within the scope of the present disclosure. Furthermore, the appended claims of this application are intended to cover all changes and modifications that fall within the scope and boundary of the appended claims, or the equivalents of this scope and boundary.

What is claimed is:

1. A method for extracting somatic mutations from single-cell transcriptome sequencing data, comprising:
processing single-cell transcriptome raw sequencing data with a first comparison and identification method to obtain a plurality of first somatic mutation sites;
processing the single-cell transcriptome raw sequencing data with a second comparison and identification method to obtain a plurality of second somatic mutation sites;
integrating the plurality of first somatic mutation sites and the plurality of second somatic mutation sites to obtain a plurality of candidate somatic mutation sites; and
performing mutation screening on the plurality of candidate somatic mutation sites to obtain final somatic mutation sites.

2. The method according to claim 1, wherein:
processing the single-cell transcriptome raw sequencing data with the first comparison and identification method to obtain the plurality of first somatic mutation sites includes:
comparing the single-cell transcriptome raw sequencing data with reference genome data using a comparison mode to obtain a comparison information record file;
labeling the comparison information record file; and
performing correction and annotation the comparison information record file that has been labeled to obtain the plurality of first somatic mutation sites; and
the correction includes sequence correction and base quality correction, and the annotation includes annotation of functional effects on encoded proteins and annotation of a database for germline mutations and RNA editing.

3. The method according to claim 1, wherein processing the single-cell transcriptome raw sequencing data with the second comparison and identification method to obtain the plurality of second somatic mutation sites includes:
comparing the single-cell transcriptome raw sequencing data with reference genome data using a comparison mode to obtain comparison results; and
obtaining the plurality of second somatic mutation sites with an identification mode from the comparison results.

4. The method according to claim 1, wherein integrating the plurality of first somatic mutation sites and the plurality of second somatic mutation sites to obtain the plurality of candidate somatic mutation sites includes:
filtering the plurality of first somatic mutation sites to obtain a plurality of filtered first somatic sites; and
comparing the plurality of filtered first somatic mutation sites with the plurality of second somatic mutation sites to obtain the plurality of candidate somatic mutation sites.

5. The method according to claim 4, wherein filtering the plurality of first somatic mutation sites includes:
excluding mutation sites, located in a preset exclusion region, of the first somatic mutation sites; and
performing site screening with the database after annotating remaining first somatic mutation sites to obtain the filtered first somatic mutation sites.

6. The method according to claim 4, wherein comparing the plurality of filtered first somatic mutation sites with the plurality of second somatic mutation sites to obtain the plurality of candidate somatic mutation sites includes, for each single cell:
regarding sites shared by the plurality of filtered first somatic mutation sites and the plurality of second somatic mutation sites as the candidate somatic mutation sites; and
regarding sites only existing in the plurality of filtered first somatic mutation sites or the plurality of second somatic mutation sites as noise sites.

7. The method according to claim 1, wherein performing mutation screening on the plurality of candidate somatic mutation sites to obtain the final somatic mutation sites includes:
screening the candidate somatic mutation sites and noise sites by using a first quality condition, a second quality condition, and a reproduction condition; and
regarding one or more of the candidate somatic mutation sites that meet all of the first quality condition, the second quality condition, and the reproduction condition as the final somatic mutation sites.

8. The method according to claim 7, further comprising:
regarding one or more of the noise sites that do not meet any of the first quality condition, the second quality condition, and the reproduction condition as final noise sites; and
regarding remaining one or more of the candidate somatic mutation sites and remaining one or more of the noise sites as undetermined candidate sites.

9. The method according to claim 8, further comprising:
using the final somatic mutation sites and the final noise sites as training data to train a mutation extraction model; and
making a prediction on the undetermined candidate sites with the mutation extraction model that has been trained to screen additional somatic mutation sites from the undetermined candidate sites.

10. The method according to claim 9, wherein the mutation extraction model includes:
a first logistic regression model established by using a detection quality value of a mutation site, coverage of reads, a probability of each genotype after normalization, a number of bases supported by each of two bases, and a ratio of mutant alleles to a number of all reads at the mutation site; and
a second logistic regression model established by using a mutation type of the mutation site, information of one base before and after the mutation site, and information of a mutation spectrum.

11. The method according to claim 10, wherein output results of the first logistic regression model and the second logistic regression model are integrated with following formula to obtain a prediction result of the mutation extraction model:
P(pos_{classifier})=½Σ_{ΣPϵ(P}_{_{seq}}_{,P}_{_{qual}}₎wP
wherein:
w denotes an integration coefficient, w=1 when P≥0.5, and w=0 when P<0.5;
P( ) represents a probability function that a candidate site is a real mutation;
pos_{classifier} represents the candidate site;
P_{qual} and P_{seq} represent an output result of the first logistic regression model and an output result of the second logistic regression model, respectively, for a same candidate mutation site.

12. An electronic device comprising:
a memory storing executable instructions; and
a processor configured to execute the instructions to:
process single-cell transcriptome raw sequencing data with a first comparison and identification method to obtain a plurality of first somatic mutation sites;
process the single-cell transcriptome raw sequencing data with a second comparison and identification method to obtain a plurality of second somatic mutation sites;
integrate the plurality of first somatic mutation sites and the plurality of second somatic mutation sites to obtain a plurality of candidate somatic mutation sites; and
perform mutation screening on the plurality of candidate somatic mutation sites to obtain final somatic mutation sites.

13. The device according to claim 12, wherein:
the processor is further configured to execute the instructions to:
compare the single-cell transcriptome raw sequencing data with reference genome data using a comparison mode to obtain a comparison information record file;
label the comparison information record file; and
perform correction and annotation the comparison information record file that has been labeled to obtain the plurality of first somatic mutation sites; and
the correction includes sequence correction and base quality correction, and the annotation includes annotation of functional effects on encoded proteins and annotation of a database for germline mutations and RNA editing.

14. The device according to claim 12, wherein the processor is further configured to execute the instructions to:
compare the single-cell transcriptome raw sequencing data with reference genome data using a comparison mode to obtain comparison results; and
obtain the plurality of second somatic mutation sites with an identification mode from the comparison results.

15. The device according to claim 12, wherein the processor is further configured to execute the instructions to:
filter the plurality of first somatic mutation sites to obtain a plurality of filtered first somatic sites; and
compare the plurality of filtered first somatic mutation sites with the plurality of second somatic mutation sites to obtain the plurality of candidate somatic mutation sites.

16. The device according to claim 15, wherein the processor is further configured to execute the instructions to:
exclude mutation sites, located in a preset exclusion region, of the first somatic mutation sites; and
perform site screening with the database after annotating remaining first somatic mutation sites to obtain the filtered first somatic mutation sites.

17. The device according to claim 15, wherein the processor is further configured to execute the instructions to, for each single cell:
regard sites shared by the plurality of filtered first somatic mutation sites and the plurality of second somatic mutation sites as the candidate somatic mutation sites; and
regard sites only existing in the plurality of filtered first somatic mutation sites or the plurality of second somatic mutation sites as noise sites.

18. The device according to claim 12, wherein the processor is further configured to execute the instructions to:
screen the candidate somatic mutation sites and noise sites by using a first quality condition, a second quality condition, and a reproduction condition; and
regard one or more of the candidate somatic mutation sites that meet all of the first quality condition, the second quality condition, and the reproduction condition as the final somatic mutation sites.

19. The device according to claim 18, wherein the processor is further configured to execute the instructions to:
regard one or more of the noise sites that do not meet any of the first quality condition, the second quality condition, and the reproduction condition as final noise sites; and
regard remaining one or more of the candidate somatic mutation sites and remaining one or more of the noise sites as undetermined candidate sites.

20. A computer-readable storage medium storing computer-executable instructions that, when executed by a processor, cause the processor to:
process single-cell transcriptome raw sequencing data with a first comparison and identification method to obtain a plurality of first somatic mutation sites;
process the single-cell transcriptome raw sequencing data with a second comparison and identification method to obtain a plurality of second somatic mutation sites;
integrate the plurality of first somatic mutation sites and the plurality of second somatic mutation sites to obtain a plurality of candidate somatic mutation sites; and
perform mutation screening on the plurality of candidate somatic mutation sites to obtain final somatic mutation sites.
